(19) United States
(12) Patent Application Publication
(10) Pub. No.: US 2011/0117136 A1
(43) Pub. Date: May 19, 2011

(54) DEHYDROEPIANDROSTERONE PRODUCTION PROMOTER AND USE THEREOF

(75) Inventors: Masayuki YAGI, Kyoto (JP); Hiroshige KAWAI, Kyoto (JP); Motoki KUBO, Kyoto (JP); Yoshikazu YONEI, Tokyo (JP); Nobuyasu MATSUURA, Okayama (JP)

(73) Assignee: ARKRAY, INC., Kyoto-shi (JP)

(21) Appl. No.: 13/013,269

(22) Filed: Jan. 25, 2011

Related U.S. Application Data
(62) Division of application No. 12/307,667, filed on Jan. 6, 2009.

(30) Foreign Application Priority Data
Mar. 20, 2007 (JP) 2007-073415

(51) Int. Cl. A61K 36/87; A61K 36/734; A61K 36/78; A61P 3/04; A61P 3/10; A61P 9/10; A61P 19/10; A61P 37/04; A61P 25/00; A61P 43/00; A61P 17/00

(52) U.S. Cl. 424/278.1; 424/765; 424/764; 424/766

(57) ABSTRACT

A new orally administerable DHEA production promoter is provided. A composition containing an extract of at least one selected from the group consisting of plants of Rosaceae Crataegus, Saururaceae Houttuynia, Vitaceae Vitis, and Compositae Anthemis is provided as a DHEA production promoter. The extract may be of one of the plants or extracts of two or more of them may be used in combination. A specific example is preferably a mixture of extracts of all four of the aforementioned plants. This DHEA production promoter also can be used as, for example, an anti-aging agent such as a skin improver as well as a pharmaceutical agent such as an immunostimulator, an antidiabetic agent, an anti-osteoporosis agent, an antiarteriosclerotic agent, an anti-obesity agent, a sleep-promoting agent, and a central nervous system depressant.

This application is a division of U.S. application Ser. No. 12/307,667, filed Jan. 6, 2009, which is a U.S. National Stage application of International Application No. PCT/JP2008/051421, filed Jan. 30, 2008, which application is incorporated herein by reference.

### TECHNICAL FIELD

The present invention relates to dehydroepiandrosterone production promoters and uses thereof.

### BACKGROUND ART

Dehydroepiandrosterone (hereinafter referred to as “DHEA”) is a sex steroid hormone that is secreted from the adrenal cortex. At least 99% thereof become sulfide in the blood and are present as dehydroepiandrosterone sulfate (hereinafter referred to as “DHEA-s”). Generally, these DHEA and DHEA-s are referred to as adrenal androgen (hereinafter the both together also may be referred to as “DHEA”). The DHEA is a sex hormone that varies over time according to age. It is known to be low before puberty, to reach a peak during puberty, and to be reduced thereafter.

Examples of efficacies of the DHEA include promotion of cornification of skin epidermis, promotion of the barrier effect, and prevention of degradation of skin wrinkles, radiance, sag, etc. It has been reported that the DHEA is effective for anti-aging. In addition, for example, an immunostimulatory effect, an antidiabetic effect, an antiosteoporotic effect, an anti-atherogenic effect, an anti-obesity effect, and an anti-central nervous system effect have been reported. Accordingly, DHEA is considered to be useful for prevention or improvement of anti-aging and various diseases, and for instance, a direct method in which DHEA is administered or an indirect method in which DHEA production is promoted are practiced. Examples of the former direct method include a method of administering DHEA and a method in which a precursor (diosgenin) of DHEA is administered and DHEA is produced from the precursor by in vivo metabolism (Patent Document 1). Furthermore, an example of the latter indirect method that has been disclosed is a method of promoting DHEA production by allowing a composition composed of rose oil, valerian oil, dimethoxymethylbenzene, and linalool to be absorbed percutaneously through nasal mucosa or skin (Patent Document 2). Moreover, it is suggested that since intake of fish scale powder results in an increase in DHEA-s metabolite (17-KS-S) in urine, fish scale powder can be used as a DHEA production promoter (Patent Document 3).

However, direct administration of DHEA, a sex hormone, or a precursor thereof results in a risk of fooling the hormone system. On the other hand, in the case of a DHEA production promoter, a sufficient amount thereof is difficult to be taken by the aforementioned percutaneous absorption. Furthermore, in the case of fish scale powder, oral use thereof is conceivable but there is a problem in flavor.

### [Patent Document 1] JP 2007-16013 A

### [Patent Document 2] JP 2007-8862 A

### [Patent Document 3] JP 2005-30671 A

### DISCLOSURE OF INVENTION

The present invention, therefore, is intended to provide a new orally administerable DHEA production promoter.

In order to achieve the aforementioned object, the DHEA production promoter of the present invention is characterized by containing an extract of at least one selected from the group consisting of plants of Rosaceae Crataegus, Saururaceae Houttuynia, Vitaceae Vitis, and Compositae Anthemis. In the present invention, “DHEA” includes not only DHEA but also DHEA-s.

The DHEA production promoter of the present invention makes it possible to increase the amount of DHEA that is produced in vivo, by oral administration thereof. As described above, since DHEA has various efficacies, intake of the DHEA production promoter of the present invention improves DHEA production. Accordingly, various effects such as anti-aging, skin improvement, and immunostimulation can be obtained. Furthermore, all the extracts contained in the DHEA production promoter of the present invention are derived from plants and also are known as herbs. Therefore it has an excellent flavor. Thus, the DHEA production promoter of the present invention can be considered to be very useful in the fields of, for example, health foods, cosmetics, and pharmaceutical agents.

### BRIEF DESCRIPTION OF DRAWINGS

FIG. 1 is a graph showing a change in blood DHEA-s over time following test meal intake in an example of the present invention.

FIG. 2 is a graph showing a change in skin elasticity index over time following test meal intake and the rate of change thereof in the aforementioned example of the present invention.

### BEST MODE FOR CARRYING OUT THE INVENTION

As described above, the DHEA production promoter of the present invention is characterized by containing an extract of at least one selected from the group consisting of plants of Rosaceae Crataegus, Saururaceae Houttuynia, Vitaceae Vitis, and Compositae Anthemis. In the present invention, it may contain an extract of any one of the aforementioned plants or may contain extracts of at least two of them. In the present invention, a preferable specific example contains an extract mixture of Rosaceae Crataegus, Saururaceae Houttuynia, Vitaceae Vitis, and Compositae Anthemis.

Examples of the plants of Rosaceae Crataegus that are used in the present invention include Crataegus oxyacantha L. and C. cuneata Sieb. et Zucc. The extract of Rosaceae Crataegus may be an extract of any one of, for example, flower, spike, pericarp, fruit, stem, leaf, branch, branches and leaves, trunk, bark, rhizome, root bark, root, and seed. The extract may be an extract of one part, extracts of at least two parts, or an extract of the entire plant. In this manner, the part from which the extract is obtained is not limited. An example thereof is an extract of fruit.

Examples of the plants of Saururaceae Houttuynia that are used in the present invention include Houttuynia cordata Thunberg. The extract of Saururaceae Houttuynia may be an extract of any one of, for example, flower, spike, pericarp, fruit, stem, leaf, rhizome, root bark, root, and seed. The extract may be an extract of one part, extracts of at least two parts, or an extract of the entire plant. In this manner, the part from which the extract is obtained is not limited. Examples thereof include extracts of above ground parts such as flower, spike, pericarp, fruits, stem, leaf, branch, branches and leaves, trunk, and arbores.

Examples of the plants of Vitaceae Vitis that are used in the present invention include Vitis vinifera L., Vitis labrusca L., V. saccharifera Makino, V. ficifolia Bunge var. lobata (Regel) Nakai, V. flexuosa Thunb., V. coiguetiae Pulliat, and V. labruscana Bailey. The extract of Vitaceae Vitis may be an extract of any one of, for example, flower, spike, pericarp, fruit, stem, leaf, branch, branches and leaves, trunk, bark, rhizome, root bark, root, and seed. The extract may be an extract of one part, extracts of at least two parts, or an extract of the entire plant. In this manner, the part from which the extract is obtained is not limited. An example thereof is an extract of the leaf.

Examples of the plants of Compositae Anthemis (Chamaemelum) that are used in the present invention include Anthemis nobilis L. (=Chamaemelum nobile). The extract of Compositae Anthemis may be an extract of any one of, for example, flower, spike, pericarp, fruit, stem, leaf, rhizome, root bark, root, and seed. The extract may be an extract of one part, extracts of at least two parts, or an extract of the entire plant. In this manner, the part from which the extract is obtained is not limited. An example thereof is an extract of anthodium.

The extract that is used in the present invention can be obtained from, for example, a desired part of one of the aforementioned plants or the whole of the plant. The extraction method is not limited, and examples thereof include a compression method and a solvent extraction method. An extraction solvent used in the solvent extraction method is not limited, and examples thereof include aqueous solvents such as water and organic solvents. Examples of the organic solvents include: lower alcohols such as ethanol and methanol; absolute ethanol; polyhydric alcohols such as propylene glycol and 1,3-butylene glycol; ketones such as acetone; esters such as acetic acid ethyl ester; as well as diethyl ether, dioxane, acetonitrile, xylene, benzene, and chloroform. The extraction solvent may be a mixture of the aforementioned aqueous solvent and organic solvent, and examples thereof include various aqueous alcohol solutions. A specific example thereof is an aqueous ethanol solution. The ratio of the organic solvent in the mixture is, for example, 5 to 80 vol%. One of the solvents may be used or two or more of them may be used in combination.

The extraction method can be carried out by, for example, using a desired part of one of the aforementioned plants or the whole of the plant as a raw material and immersing it in the aforementioned extraction solvent. For example, the raw material can be immersed directly in the extraction solvent or may be pulverized and then may be immersed in the extraction solvent. When using two raw materials, each of them may be subjected to an extraction treatment, or a mixture of two or more raw materials may be subjected to an extraction treatment. When using two or more raw materials, the ratios of the raw materials to be added are not limited but are, for example, equivalent (weight) to each other. When using the aforementioned four plants, it is preferable that a raw material mixture obtained by mixing the respective raw materials at 1:1:1:1 (dry weight ratio) be subjected to an extraction treatment.

The ratios of the raw material and the extraction solvent to be added are not limited. With respect to 100 g of raw material, the extraction solvent is, for example, 0.1 to 1000 L and preferably 1 to 100 L. The time for immersing the raw material in the extraction solvent is not limited. It can be set suitably according to, for example, the type of the plant, the amount of the plant, as well as the type and amount of the extraction solvent. For example, when 100 g of raw material is immersed in 10 L of extraction solvent, the time is preferably at least 0.5 hour and more preferably 0.5 to 24hours.

The extraction condition is not limited. However, for example, in the case of extraction using an aqueous solvent such as water, hot water extraction is preferable. Furthermore, it is preferable that a raw material be immersed beforehand in the aqueous solvent under the aforementioned condition prior to the hot water extraction. The heating temperature that is employed for the hot water extraction is not limited and is, for example, at least 30° C. and preferably 50 to 100° C. Furthermore, the treating time for hot water extraction can be set suitably according to, for example, the type or amount of the raw material to be treated, or the amount of the extraction solvent. For instance, when 100 g of raw material is subjected to extraction with 10 L of extraction solvent, the treating time is preferably at least 0.5 hour and more preferably 0.5 to 24 hours.

The resultant extract may be used, for example, directly for a DHEA production promoter or further may be subjected to a purification treatment and then may be used for a DHEA production promoter. The purification treatment is not limited and examples thereof include a distillation treatment, filtration treatment, chromatography treatment, and drying treatment.

The form of the extract according to the present invention is not limited and it may be, for example, in the form of a solution, paste or powder. It can be selected suitably according to the form of the DHEA production promoter described later.

The DHEA production promoter of the present invention is not limited by the form thereof as long as it contains the aforementioned extract. It may be, for example, in the form of a solid, liquid, or a gel. Specific examples of the form include ampule, capsule, pill, tablet, powder, and granule. Furthermore, the DHEA production promoter of the present invention may contain, for example, various additives such as diluents in addition to the extract.

As described above, the DHEA production promoter of the present invention contains, as an active ingredient for the DHEA production promotion, an extract of at least one selected from the group consisting of plants of Rosaceae Crataegus, Saururaceae Houttuynia, Vitaceae Vitis, and Compositae Anthemis. Accordingly, it is obvious that the DHEA production promoter also is excellent in safety. The intake of the DHEA production promoter of the present invention is not limited and can be set suitably according to, for example, sex, age, and type of the disease. However, in the case of a healthy adult, the intake per day is preferably 10 to 2000 mg of the aforementioned extract (solid content) and more preferably 50 to 1000 mg. The intake method also is not limited and can be determined according to, for example, the form of the DHEA production promoter of the present invention. For instance, it is possible to take it as a drink or a supplement, or by mixing it with another food.

Next, the anti-aging agent of the present invention is characterized by containing the aforementioned DHEA production promoter of the present invention. The present invention makes it possible to improve or retard, for example, aging-related and senescence-related phenomena. As long as the anti-aging agent of the present invention contains a DHEA production promoter of the present invention, no other limitations are posed on, for example, the composition and constitution thereof. For example, the similar composition and form to those of the aforementioned DHEA production promoter of the present invention can be employed.

The skin improver of the present invention is characterized by containing a DHEA production promoter of the present invention described above. The present invention makes it possible to, for example, prevent cornification of the skin, prevent wrinkles, radiance, sag, etc., and maintain or improve elasticity of the skin. As long as the skin improver of the present invention contains a DHEA production promoter of the present invention, no other limitations are posed on, for example, the composition and constitution thereof. For instance, the similar composition and form to those of the aforementioned DHEA production promoter of the present invention can be employed.

Compositions containing DHEA production promoters of the present invention (i.e. compositions containing the aforementioned extracts) can be used as, for example, various pharmaceutical agents. Since DHEA exhibits an immunostimulatory effect, an antidiabetic effect, an antiosteoporotic effect, an anti-atherogenic effect, an anti-obesity effect, a sleep promotion effect, and an anti-central nervous system effect, examples of the pharmaceutical agents include an immunostimulator, an antidiabetic agent, an anti-osteoporosis agent, an antiarteriosclerotic agent, an anti-obesity agent, a sleep-promoting agent, and a central nervous system depressant. As long as the pharmaceutical agents of the present invention contain DHEA production promoters of the present invention, for example, the compositions or constitutions thereof are not limited and can be similar compositions or forms to those of the aforementioned DHEA production promoters of the present invention.

Next, a method for promotion of DHEA of the present invention is characterized by including administering a DHEA production promoter of the present invention.

In the present invention, the subject to which the aforementioned production promoter is to be administered is not particularly limited. Examples thereof include mammals including human and it is preferably human. Furthermore, the method of administering the DHEA production promoter also is not particularly limited. However, since the effect can be obtained even by oral administration, oral administration is preferable. In this case, for example, the intake is as described above.

Next, the method of anti-aging of the present invention is an anti-aging method that utilizes DHEA production promotion, wherein the method for the aforementioned production promotion is a DHEA production promotion method of the present invention. In the present invention, as long as a DHEA production promoter of the present invention is administered, specific conditions therefor are not particularly limited. It can be carried out in the same manner as in the case of the aforementioned production promotion method.

Next, the method of skin improving of the present invention is a skin improving method that utilizes DHEA production promotion, wherein the method for the aforementioned production promotion is a DHEA production promotion method of the present invention. In the present invention, as long as a DHEA production promoter of the present invention is administered, specific conditions therefor are not particularly limited. It can be carried out in the same manner as in the case of the aforementioned production promotion method.

Next, a method of preventing or treating a disease according to the present invention is a method of preventing or treating a disease by DHEA production promotion, wherein the method for the aforementioned production promotion is a DHEA production promotion method of the present invention. In the present invention, as long as a DHEA production promoter of the present invention is administered, specific conditions therefor are not particularly limited. It can be carried out in the same manner as in the case of the aforementioned production promotion method.

As described above, the disease is not particularly limited but is, for example, a disease caused by a DHEA decrease. Specific examples thereof include diabetes, osteoporosis, arteriosclerosis, and obesity. Furthermore, since DHEA production promotion results in, for example, immunostimulation, central nervous system action, and sleep promotion, examples thereof also include diseases that can be improved thereby.

Next, examples of the present invention are described. However, the present invention is not limited by the following examples.

### Example 1

The respective dried products of Anthemis nobilis L. (anthodium), Houttuynia cordata Thunberg (above ground part), Crataegus oxyacantha L. (fruit), and Vitis vinifera L. (leaf) are mixed in equal amounts (weight) to each other. This dry mix (100 g) was immersed in purified water (10 L) at approximately 80° C. for about five hours and thereby a plant extract of the dry mix was extracted. This extract was filtered and thereby a residue was removed. Thus, a filtrate (about 10 kg) was recovered. This filtrate further was dried and thereby the solvent (purified water) was removed. Thus 20 g of powder was obtained. This powder was mixed with diluents (dextrin, starch, calcium stearate, silicon dioxide, caramel pigment, titanium dioxide, and lecithin derived from soybean) and this was prepared in hard capsules made of gelatin derived from porcine so that 240 mg of the aforementioned powder (solid content) was contained per three capsules. Three capsules composed one test meal.

The subjects were six adult males who were type 2 diabetic patients and one female who was a type 2 diabetic patient (seven patients in total). The subjects took the aforementioned test meal once a day for 12 weeks. With the start of intake being considered as Day 0, blood was collected on Day 0 as well as in the 8^{th} week and the 12^{th} week, and the blood DHEA-s was measured by the method described below. Furthermore, the skin elasticity index was measured by the method described below on Day 0 as well as in the 2^{nd} week, the 4^{th} week, the 8^{th} week, and the 12^{th} week. All the subjects refrained from, for example, surfeit and extreme exercise and maintained their lifestyle during the test period. For blood collection, the subjects stayed off any intake other than water from 10 pm on the previous day to completion of the blood collection, and blood was collected between 9 am and 12 pm on the day of the blood collection. The DHEA-s was measured immediately after blood collection.

The blood DHEA-s was measured using a commercially available kit (DPC DHEA-S kit (trade name), manufactured by IATRON LAB INC.) according to the protocol thereof. The skin elasticity index was measured using a Cutometer (trade name) (Integral Corporation) according to the protocol thereof. These measurement results were indicated in average value±standard deviation. Microsoft Office Excel 2003 (trade name, manufactured by Microsoft Corporation) was used to tally data and Dr. SPSSII for Windows (registered trademark) (trade name, manufactured by SPSS Inc.) was used for statistical analysis.

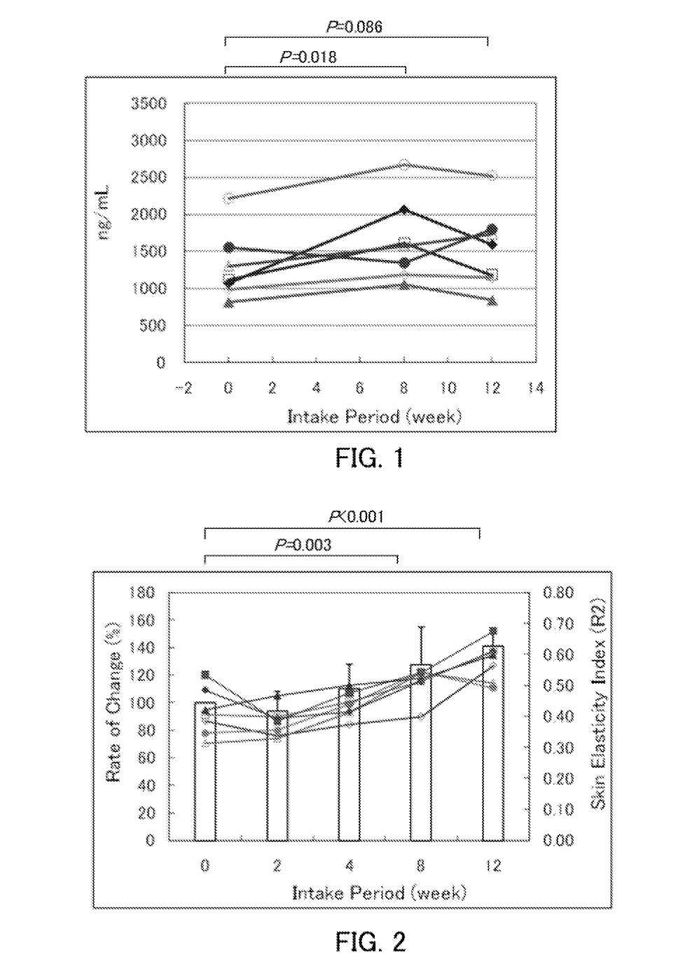

The results are shown in FIGS. 1 and 2. FIG. 1 is a graph showing a change in blood DHEA-s over time following test meal intake. FIG. 2 is a graph showing a change in skin elasticity index over time following test meal intake and a rate of the change. In FIG. 2, the line graph indicates the elasticity index, while the bar graph indicates the rate of change in elasticity index.

As shown in FIG. 1, it was proved that intake of the test meals improved the blood DHEA-s of each subject. This result shows that the intake of a DHEA production promoter of the present invention promotes production of DHEA. As shown in FIG. 2, the intake of test meals improved the skin elasticity index of each subject and the rate of change (average) thereof also improved. These results show that the intake of a DHEA production promoter of the present invention promotes production of DHEA and that skin elasticity is improved accordingly. It has been confirmed that the aforementioned extract has DPPH radical scavenging activity that is ten times that of a-tocopherol and super oxide scavenging activity that is 95 times that of a-tocopherol. Furthermore, it also has been proved that the aforementioned extract has an antioxidant effect.

### Industrial Applicability

As described above, the present invention makes it possible to increase the amount of DHEA that is produced in vivo, by oral administration. As described above, since DHEA has various efficacies, intake of a DHEA production promoter of the present invention improves DHEA production and thereby various effects such as anti-aging, skin improvement, and immunostimulation can be obtained. Furthermore, all the extracts contained in the DHEA production promoters of the present invention are derived from plants and also are known as herbs. Therefore they are excellent in flavor. Accordingly, the DHEA production promoters of the present invention are very useful in the fields of, for example, health foods, cosmetics, and pharmaceutical agents.

1.-16. (canceled)

17. A method for promoting dehydroepiandrosterone production in a subject, comprising:
administering, to the subject, a composition that comprises an amount of extracts of at least two selected from the group consisting of plants of Rosaceae Crataegus, Saururaceae Houttuynia, Vitaceae Vitis, and Compositae Anthemis effective for promoting dehydroepiandrosterone production.

18. The method according to claim 17, wherein the amount is effective in preventing aging in the subject.

19. The method according to claim 17, wherein the amount is effective in improving skin in the subject.

20. The method according to claim 17, wherein the plant of Rosaceae Crataegus is Crataegus oxyacantha L., the plant of Saururaceae Houttuynia is Houttuynia cordata Thunberg, the plant of Vitaceae Vitis is Vitis vinifera L., and the plant of Compositae Anthemis is Anthemis nobilis L.

21. The method according to claim 17, wherein the composition comprises an extract mixture of a plant of Rosaceae Crataegus, a plant of Saururaceae Houttuynia, a plant of Vitaceae Vitis, and a plant of Compositae Anthemis.

22. The method according to claim 17, wherein the amount of the extracts administered is 10 to 2000 mg of the extracts per day.

23. The method according to claim 17, wherein the amount of the extracts administered is 50 to 1000 mg of the extracts per day.

24. A method for treating or preventing a disease affected by dehydroepiandrosterone production in a subject, comprising:
administering, to the subject, a composition that comprises an amount of extracts of at least two selected from the group consisting of plants of Rosaceae Crataegus, Saururaceae Houttuynia, Vitaceae Vitis, and Compositae Anthemis effective for promoting dehydroepiandrosterone production.

25. The method according to claim 24, wherein the disease is at least one selected from the group consisting of diabetes, osteoporosis, arteriosclerosis, and obesity.

26. The method according to claim 24, wherein the amount is effective in providing immunostimulation in the subject, providing central nervous system action in the subject, or promoting sleep in the subject.

27. The method according to claim 24, wherein the plant of Rosaceae Crataegus is Crataegus oxyacantha L., the plant of Saururaceae Houttuynia is Houttuynia cordata Thunberg, the plant of Vitaceae Vitis is Vitis vinifera L., and the plant of Compositae Anthemis is Anthemis nobilis L.

28. The method according to claim 24, wherein the composition comprises an extract mixture of a plant of Rosaceae Crataegus, a plant of Saururaceae Houttuynia, a plant of Vitaceae Vitis, and a plant of Compositae Anthemis.

29. The method according to claim 24, wherein the amount of the extracts administered is 10 to 2000 mg of the extracts per day.

30. The method according to claim 24, wherein the amount of the extracts administered is 50 to 1000 mg of the extracts per day.
